(19) United States
(12) Patent Application Publication
(10) Pub. No.: US 2004/0115263 A1
(43) Pub. Date: Jun. 17, 2004

(54) Use of bupropion for treating restless legs syndrome

(76) Inventors: David W. Robertson, Glenview, IL (US); Grant Krafft, Glenview, IL (US)

Correspondence Address: PHARMACIA & UPJOHN, 301 HENRIETTA ST, 0228-32-LAW, KALAMAZOO, MI, 49007, US

(21) Appl. No.: 10/645,649

(22) Filed: Aug. 20, 2003

Related U.S. Application Data
(60) Provisional application No. 60/405,943, filed on Aug. 26, 2002.

(51) Int. Cl. A61K 9/22; A61K 31/137

(52) U.S. Cl. 424/468; 514/649

(57) ABSTRACT

The present invention provides for use of bupropion or a pharmaceutically acceptable salt thereof in the treatment of restless legs syndrome in humans.

### CROSS-REFERENCE TO OTHER APPLICATIONS

This application claims the benefit of U.S. Provisional Application Serial No. 60/405,943 filed on Aug. 26, 2002.

### FIELD OF THE INVENTION

The present invention relates generally to treatment of restless legs syndrome and particularly to use of bupropion and the pharmacologically acceptable salts thereof in the treatment of restless legs syndrome.

### BACKGROUND OF THE INVENTION

Restless legs syndrome (RLS) is a distinctive clinical syndrome and one of the most common neurological disorders with a prevalence of about 5-10% in the general population. There appears to be two forms of restless legs syndrome: the idiopathic and the uremic form. The term “restless legs syndrome” or “RLS” as used herein refers to both idiopathic and the uremic forms of RLS. The characteristics of RLS are sensory and motor symptoms that are evoked by rest, either quiet wakefulness or attempts to sleep. Patients with RLS have unpleasant sensations in the legs and an uncontrollable urge to move when at rest in an effort to relieve these feelings. RLS sensations are often described by people as burning, creeping, tugging, or like insects crawling inside the legs. Often called paresthesias (abnormal sensations) or dysesthesias (unpleasant abnormal sensations), the sensations range in severity from uncomfortable to irritating to painful. The most distinctive or unusual aspect of the condition is that lying down and trying to relax activates the symptoms.

More than 80 percent of people with RLS also experience a condition known as periodic limb movement disorder (PLMD). PLMD is characterized by involuntary leg twitching or jerking movements during sleep that typically occur every 10 to 60 seconds, sometimes throughout the night. The symptoms cause repeated awakening and severely disrupted sleep. Unlike RLS, the movements caused by PLMD are involuntary-people have no control over them. Although many patients with RLS also develop PLMD, most people with PLMD do not experience RLS. Like RLS, the cause of PLMD is unknown.

RLS is extensively described in Karin Stiasny, et al.: Clinical symptomatology and treatment of restless legs syndrome and periodic limb movement disorder. Sleep Medicine Review. Vol. 6, No. 4, pp 253-265, 2002, and in references cited in U.S. Pat. Nos. 6,001,861 and 6,114,326, incorporated herein by reference.

Clinical diagnostic criteria for RLS have been established by the International RLS Study Group (IRLSSG). They consist of four minimal criteria based solely on the patient's history. These are: (1) a desire to move the limbs, usually associated with paresthesias/dysesthesias; (2) motor restlessness (i.e. rubbing the legs, tossing and turning in bed, stretching and flexing the legs, or pacing the floor) (3); symptoms or exclusive presence of symptoms at rest (i.e. lying, sitting) with at least partial or temporary relief by activity; and (4) worsening of symptoms during the evening or night. Additional features such as sleep disturbances, involuntary nocturnal periodic limb movements, a progressive clinical course, an unremarkable neurological examination in idiopathic RLS or a positive family history are frequently found in RLS but are not mandatory for diagnosis. (See, Karin Stiasny, et al.: Clinical symptomatology and treatment of restless legs syndrome and periodic limb movement disorder. Sleep Medicine Review. Vol. 6, No. 4, pp 253-265, 2002.) The severity of RLS may be quantified by the RLS Severity Scale that was recently developed and validated by the IRLSSD. See, Karin Stiasny, et al.: Clinical symptomatology and treatment of restless legs syndrome and periodic limb movement disorder. Sleep Medicine Review. Vol. 6, No. 4, pp 253-265, 2002.

There is no cure for RLS so far. Over the years various pharmacological agents have been proposed or used to treat symptoms of RLS. While one medication, Restex® (a levodopa-based product marketed by Roche Pharmaceuticals), has reportedly been approved recently in Germany for the treatment of RLS, no medication is currently approved in the United States for this indication.

The typical pharmacological agents that have been proposed or used as treatments for RLS fall into four categories: anticonvulsant drugs, benzodiazepines, opioids and dopaminergic agents.

Anticonvulsants appear to work by decreasing sensory disturbances (the unpleasant sensations) and the urge to move. These drugs are particularly effective for some, but not all, patients with marked daytime symptoms, particularly people who have pain syndromes associated with their RLS. Gabapentin (Neurontin) is the anticonvulsant that has shown the promise in treating the symptoms of RLS. Possible side effects of gabapentin include dizziness, sleepiness, fatigue, increased appetite, and unsteadiness. The sedative properties of gabapentin may impair the ability to operate heavy machinery, including a motor vehicle.

Benzodiazepines that have been used to treat RLS include clonazepam (Klonopin), nitrazepam, lorazepam and temazepam. Benzodiazepines do not fully suppress RLS sensations or leg movements, but allow patients to obtain more sleep despite the problems. Drawbacks to the use of these medications include the potential for confusion and daytime sleepness. In addition, dependency can develop with the use of all benzodiazepines and withdrawal is associated with great discomfort in patients.

Opioids, which are narcotic analgesic (pain-killing) drugs and relaxing drugs, can suppress RLS and PLMS in some people especially those with severe and relentless symptoms of RLS. Examples of medications in this category used to treat RLS include codeine, propoxyphene (Darvon or Darvocet), oxycodone (Percocet, Tylox, Roxiprin), pentazocine (Talwin), hydrocodone (Vicodin), and methadone. Side effects and adverse reactions include dizziness, sedation, nausea, vomiting, constipation, hallucination, and headache. In addition, the use of opioids carries the risk of abuse and addiction.

Dopaminergic drugs are considered the first line of pharmacological treatment for RLS. These drugs are usually used to treat Parkinson's disease, a condition different and distinct from RLS. Examples of drugs in this category used to treat RLS include L-dopa, bromocriptine, and pergolide. Several studies have shown that L-dopa given with a peripheral carboxylase inhibitor at a 10:1 ratio is effective in treating RLS. See for example the following articles: Brodeur C, Montplaisir J, Marinier R, Godbout R., “Treatment of RLS and PMS with L-dopa: a double-blind controlled study,” Neurology; 35:1845-1848 (1988). Montplaisir J, Godbout R, Poirier G, Bedard M. A., “Restless legs syndrome and periodic movements in sleep: physiopathology and treatment with L-dopa,” Clinical Neuropharmacology; 9:456-463 (1986). Von Scheele C, “Levodopa in restless legs,” Lancet; 2:426-427 (1986). Akpinar S., “Restless legs syndrome treatment with dopaminergic drugs,” Clinical Neuropharmacology; 10:69-79 (1987). Two significant and common problems with the use of L-dopa have been noted: 1) the short half-life of the drug, compounded by the tendency of systems to recur later in the night after initial response to treatment, often leads to poor sleep quality and 2) the development of rebound of symptoms and augmentation. Augmentation is the tendency for systems to develop earlier in the day and to be more severe than the systems that occurred before treatment with L-dopa began. Augmentation is the most serious, and common, complication with L-dopa therapy. Recent experience suggests that augmentation can be a complicating feature in 65% to 80% of cases. In addition, when L-dopa treatment is repeated in the middle of the night, patients with severe cases may experience de novo paraesthesia and restlessness during the daytime.

Bromocriptine, a D2 receptor agonist, was also used in RLS treatment. Walters, A S; Hening, W A; Chokroverty, S; Gidro-Franck, S. A double blind randomized crossover trial of bromocriptine and placebo in restless leg syndrome. Ann Neurol; 1988 24:455-458. Side effects reported were transient nasal stuffiness and lightheadedness in one patient.

Pergolide, a dopamine D1/D2 agonist, in combination with a low dose of L-dopa can lead to clinical improvement in patients who do not respond to L-dopa alone, but can also cause several important side effects such as orthostatic hypotension and gastrointestinal problems and augmentation.

Non-pharmacological therapies have also been used or suggested for treating RLS, such as improved nutrition, exercise, sleep hugiene, transcutaneous electrical nerve stimulation, conditioning therapies, and various procedures to reduce incompetent veins. None of these nonpharmacological therapies, however, has been clearly established to effective.

Fairly recent patent documents have suggested that new treatments may be available and useful but the new treatments have not yet been widely prescribed, see U.S. Pat. No. 6,114,326 which discloses the use of cabergoline, a synthetic ergoline derivative and a dopamine agonist, either by itself or in combination with levodopa as a treatment for RLS. In U.S. Pat. No. 6,001,861, the use of pramipexole a dopamine D₃/D₂ agonist to treat RLS is disclosed.

In view of the problems with all the possible treatments mentioned above, it is fair to say, there is no optimally effective treatment for RLS. The choice of where to turn for a possible treatment of RLS is a problem for any treating physician, with the possible known treatments presenting serious drawbacks. Currently a physician might be tempted to use levodopa in conjunction with a dopa decarboxylase inhibitor (DDCI) such as carbidopa. Although many RLS patients show an excellent response to levodopa, there is increasing evidence that the relatively short duration of action and augmentation of symptoms may be a limiting factor of levodopa therapy. Considering the problem of augmentation with levodopa therapy, alternative treatment options for RLS are of major interest, especially for patients with severe RLS. In view of the above, there clearly exist a need for an effective treatment of RLS.

Bupropion is the generic name for the compound 1-(3-chlorophenyl)-2[(1,1-dimethyl-ethyl)amino]-1-propanone. Structurally, bupropion exists in stereoisomers. The racemic mixture of bupropion, or (±)-bupropion, is currently commercially available for treatment of depression and for smoking cessation. The racemic mixture of bupropion which is commercially available is administered as a hydrochloride salt. Wellbutrin® is the trade name for the bupropion salt, bupropion HCl, an anti-depressant manufactured by Glaxo Wellcome. A sustained-release formulation of bupropion HCl, Wellbutrin SR®, is also indicated for the treatment of depression. Glaxo Wellcome also has FDA approval to market a sustained release formulation of bupropion HCl as an aid to smoking cessation treatment for the smoking cessation indication. Glaxo Wellcome is marketing this product under the trade name Zyban®. Zyban® can be used either alone or in combination with a nicotine transdermal system (NTS). In addition, European Patent Application No. 84101070.5 discloses the benefits of bupropion maleate over bupropion hydrochloride.

Additionally, the racemic mixture of bupropion has been disclosed for use in the treatment of the following conditions: effects of ethanol (U.S. Pat. No. 4,393,078); Tardine Dyskinesia (U.S. Pat. No. 4,425,363); Minimal Brain Dysfunction (U.S. Pat. No. 4,435,449); amelioration of prostate hypertrophy and sexual dysfunction (U.S. Pat. No. 4,835,147); psychostimulant addiction (U.S. Pat. No. 4,935,429); Psychosexual Dysfunction (U.S. Pat. No. 4,507,323); and weight gain (U.S. Pat. No. 4,895,845).

U.S. Pat. No. 6,280,763 discloses the use of optically pure (−)-bupropion for treating Parkinson's disease. U.S. Pat. No. 6,110,973 discloses the use of optically pure (−)-bupropion for treating obesity and weight gain.

Disclosed herein is use of bupropion and the pharmacologically acceptable salts thereof as treatment for restless legs syndrome.

### SUMMARY OF THE INVENTION

The present invention provides for methods for treating restless legs syndrome in a patient suffering from or susceptible to such condition comprising the administration of an effective amount of bupropion or pharmaceutically acceptable salts thereof. The present invention also provides for use of bupropion or pharmaceutically acceptable salts thereof for the preparation of a medicament useful for treating restless legs syndrome in a patient suffering from or susceptible to such condition.

The bupropion can be administered in the form of racemic mixture of bupropion (hereinafter “(±)-bupropion”), its (+) enantiomer (hereinafter “(+)-bupropion”), its (−) enantiomer (hereinafter “(−)-bupropion”), or the mixture of the (+) enantiomer and (−) enantiomer at any ratio. In a preferred embodiment, the invention is directed to methods for treating restless legs syndrome comprising the administration of an effective amount of (±)-bupropion hydrochloride. In another preferred embodiment, the invention is directed to methods for treating restless legs syndrome comprising the administration of an effective amount of (−)-bupropion or its pharmaceutically acceptable salts.

In a particular embodiment, the bupropion is administered in a composition comprising (−)-enantiomer substantially free of the (+)-enantiomer. In a preferred embodiment the bupropion is administered in a composition containing at least 90% by weight of (−)-bupropion and 10% by weight or less of (+)-bupropion. In another preferred embodiment the bupropion is administered in a composition containing approximately 99% by weight of (−)-bupropion, and 1% or less of the (+)-bupropion. In still another preferred embodiment, the bupropion is administered in a composition containing greater than 99% by weight of the (−)-enantiomer of bupropion, again based on the total amount of bupropion present.

### DETAILED DESCRIPTION OF THE INVENTION

The present invention encompasses methods for treating restless legs syndrome in a patient suffering from or susceptible to such condition comprising the administration of an effective amount of bupropion or a pharmaceutically acceptable salt thereof. Examples of the pharmaceutically acceptable salts of bupropion suitable for use in the present invention include bupropion maleate and bupropion hydrochloride.

The process of preparing bupropion is known in the art. For example, bupropion can be prepared according to the procedures described in U.S. Pat. Nos. 3,819,706 and 3,885,046. The process of preparing optically pure enantiomer of bupropion is also known in the art. For example a process of preparing (−)-enantiomer of bupropion is disclosed in U.S. Pat. No. 6,277,887. In brief, the synthesis of the (−)-isomer of bupropion may start from readily available 3-chloropropiophenone (1). Reaction of (1) with a (2R,3R)-(+)-dialkyl tartrate such as (+)-dimethyl or diethyl tartrate in the presence of an acid catalyst such as methanesulfonic acid gives the chiral acetal (2) according to Castaldi (G. Castaldi, et al., J. Org. Chem. 1987, 52: 3018). Steroselective bromination with bromine in carbon tetrachloride, or alternatively ethyl acetate, then produces the corresponding bromoacetal (3) as the major product according to the above-referenced procedure developed by Castaldi and co-workers. The bromoacetal (3) is purified by column chromatography to yield the optically pure bromoacetal (3) which is then hydrolyzed in the presence of an acid to afford the bromoketone (4). Treatment of the bromoketone (4) with tert-butylamine, followed by reaction with anhydrous hydrogen chloride, then produces optically pure (−)-bupropion hydrochloride (5) after recrystallization.

Alternatively, the optically pure isomers of bupropion can be prepared asymmetrically according to the procedures reported by Musso et al., “Synthesis and Evaluation of the Antidepressant Activity of the Enantiomers of Bupropion”, Chirality 5:495-500 (1993) which is incorporated herein by reference in its entirety.

In addition to the above-described methods the stereoisomers of bupropion may be obtained by resolutions of a mixture of enantiomers of bupropion using conventional means such as an optically active resolving agent; see, for example, “Stereochemistry of Carbon Compounds”, by E. L. Eliel (McGraw-Hill, N.Y., 1962), and S. H. Wilen, p. 268 in “Tables of Resolving Agents and Optical Resolutions” (E. L. Eliel, Ed., Univ. of Notre Dame Press, Notre Dame, Ind. 1972).

Any suitable route of administration may be employed for providing the patient with an effective dosage of bupropion. For example, oral, rectal, parenteral, transdermal, subcutaneous, intrathecal, intramuscular and the like may be employed as appropriate. The most preferred route of the present invention is the oral route. They may be conveniently presented in unit dosage form and prepared by any of the methods well-known in the art of pharmacy.

Dosage forms for bupropion in the present invention include tablets, coated tablets, cachets, capsules, troches, dispersions, sustained release formulations, suspensions, solutions, patches and the like.

Dosage forms for the present invention can be prepared by known methods suitable for preparing bupropion. In general, bupropion can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous injections or infusions). In preparing the compositions for oral dosage form, any of the usual-pharmaceutical media may be employed, for example, water, glycols, oils, alcohols, flavoring agents, preservatives, coloring agents and the like in the case of oral liquid preparations, for example, suspensions, elixirs and solutions; or aerosols; or carriers such as starches, sugars, microcrystalline cellulose, stabilizers, diluents, granulating agents, lubricants, binders, fillers, disintegrating agents and the like in the case of oral solid preparations such as, powders, capsules and tablets, with the solid oral preparations being preferred over the liquid preparations. The preferred solid oral preparation is tablets. Tablets may be prepared by compression or molding, optionally with one or more accessory ingredients. Compressed tablets may be prepared by compressing in a suitable machine the active ingredient in a free-flowing form such as powder or granules, optionally mixed with a binder, filler, lubricant, inert diluent, and/or surface active or dispersing agent. Molded tablets may be made by molding in a suitable machine a mixture of the powdered compound moistened with an inert liquid diluent. If desired, tablets may be coated by standard aqueous or nonaqueous techniques. Desirably, each tablet contains from about 10 mg to about 250 mg of the active ingredient, and-each cachet or capsule contains from about 10 mg to about 250 mg of the active ingredient. Most preferably, the tablet, cachet or capsule contains one of four dosages: about 50 mg, about 75 mg, about 100 mg and about 150 mg of active ingredient.

In addition to the common dosage forms set-forth above, the compounds of the present invention may also be administered by controlled release or sustained release means and/or delivery devices such as those described in U.S. Pat. Nos. 3,845,770; 3,916,899; 3,536,809; 3,598,123; 3,630,200, 4,008,719, 4,687,660, and 4,769,027, 5,427,798 6,210,716, the disclosures of which are hereby incorporated by reference.

The effective amount of bupropion in the treatment of RLS will vary depending various factors known to the treating physicians, such as the severity of the condition to be treated, route of administration, formulation and dosage forms, physical characters of bupropion used, and age, weight and response of the individual patients. In general, the recommended daily dose range lies within the range of from about 10 mg to about 750 mg per day, generally divided equally into doses given three or four times a day. Typically, a daily dose range should be between 50 mg- and 600 mg per day, usually divided equally into a three or four times a day dosing. More typically, a daily dose range should be between 60 mg and 450 mg per day, usually divided equally into a three times or a four times a day dosing. It may be necessary to use dosages outside these ranges in some cases. The physician will know how to increase, decrease or interrupt treatment based upon patient response.

The embodiments of the present invention described above are intended to be merely exemplary and those skilled in the art will recognize, or be able to ascertain using no more than routine experimentation, numerous equivalents to the specific procedures described herein. All such equivalents are considered to be within the scope of the present invention and are covered by the claims.

### DEFINITIONS

The term “bupropion” as used herein means the racemic mixture of bupropion (hereinafter “(±)-bupropion”), its (+) enantiomer (hereinafter “(+)-bupropion”), its (−) enantiomer (hereinafter “(−)-bupropion”), or the mixture of the (+) enantiomer and (−) enantiomer at any ratio.

The term “treating restless legs syndrome” as used herein means a relief from, alleviation of, or reduction of frequency, or severity or both, of any of the symptoms of restless legs syndrome.

The term “(−)-bupropion” as used herein means optically pure (−)-enantiomer of bupropion or bupropion composition substantially free of the (+)-s enantiomer.

The term “substantially free of the (+)-enantiomer” as used herein means that the composition contains a greater proportion of the (−)-enantiomer of bupropion in relation to the (+)-enantiomer of bupropion. These percentages are based on the total amount of bupropion present in the composition.

The term “effective amount” of bupropion or a pharmaceutically acceptable salt thereof as used herein means the amount of bupropion or a pharmaceutically acceptable salt thereof administered to a patient that is sufficient to treat restless legs syndrome in the patient.

### EXAMPLES

The following examples illustrate the preparation of compositions of the present invention. It will be apparent to those skilled in the art that many modifications, both to materials and methods, may be practiced without departing from the purpose and interest of this invention.

All temperatures-are in degrees Celsius.

### Example 1

Oral formulation (Coated Tablets)

| Formula | Quantity per Tablet (mg.) |
|---|---|
| bupropion | 75 |
| Lactose | 125 |
| Corn Starch | 5.0 |
| Water (per thousand Tablets) | 30.0 ml* |
| Magnesium Stearate | 0.5 |
| Corn Starch | 25.0 |

*The water evaporates during manufacture.

The active ingredient is blended with the lactose until a uniform blend is formed. The smaller quantity of corn starch is blended with a suitable quantity of water to form a corn starch paste. This is then mixed with said uniform blend until a uniform wet mass is formed. The remaining corn starch is added to the resulting wet mass and mixed until uniform granules are obtained. The granules are then screened through a suitable milling machine, using a ¼ inch stainless steel screen. The milled granules are then dried in a suitable drying oven until the desired moisture content is obtained. The dried granules are then milled through a suitable milling machine using ¼ mesh stainless steel screen. The magnesium stearate is then blended and the resulting mixture is compressed into tablets of desired shape, thickness, hardness and disintegration. Tablets are coated by standard aqueous or nonaqueous techniques.

### Example 2

Oral Formulation (Capsules)

| | Quantity per capsule in mg. | | |
|---|---|---|---|
| Formula | A | B | C |
| bupropion | 25 | 50 | 75 |
| Lactose | 149.5 | 124.5 | 374 |
| Corn Starch | 25 | 25 | 50 |
| Magnesium Stearate | 0.5 | 0.5 | 1.0 |
| Compression Weight | 200.0 | 200.0 | 500.0 |

The active ingredient, bupropion, lactose, and corn starch are blended until uniform; then the magnesium stearate is blended into the resulting powder. The resulting mixture is encapsulated into suitably sized two-piece hard gelatin capsules.

### Example 3

Sustained Release Oral Formulation (Tablet)

| Formula | Quantity per Tablet (mg.) |
|---|---|
| bupropion hydrochloride | 100 |
| Contramid crosslinked amylose | 98.8 |
| Cysteine hydrochloride | 7.5 |
| Magnesium stearate | 1.2 |

Bupropion Hydrochloride is formulated using Contramid® (Labopharm, Inc, Quebec) technology. The formulation is prepared by blending the ingredients above (dry) and compressing into tablets. Alternatively, the ingredients can be formulated using wet granulation technology known in the art. (See Example 1).

### Example 4

Sustained Release Oral Formulation (Tablet)

| Formula | Quantity per Tablet (mg.) |
|---|---|
| Contramid .RTM. crosslinked amylose | 98.8 |
| Cysteine hydrochloride | 7.5 |
| (−)-bupropion hydrochloride | 75 |
| Magnesium stearate | 1.2 |

(−)-Bupropion Hydrochloride is formulated using Contramid (Labopharm, Inc, Quebec), technology. The formulation is prepared by blending the ingredients above (dry) and compressing into tablets. Alternatively, the ingredients can be formulated using wet granulation technology known in the art. (See Example 1).

The embodiments of the present invention described above are intended to be merely exemplary and those skilled in the art will recognize, or be able to ascertain using no more than routine experimentation, numerous equivalents to the specific procedures described herein. All such equivalents are considered to be within the scope of the present invention and are covered by the following claims.

The contents of all references described herein are hereby incorporated by reference.

What is claimed is:

1. A method of treating restless legs syndrome in a patient, comprising administering to said patient in need of treatment a therapeutically effective amount of bupropion or pharmaceutically acceptable salts thereof.

2. The method according to claims 1, wherein bupropion is administered by intravenously, transdermally, or orally.

3. The method according to claims 1, wherein bupropion is administered in the form of tablet, cachet, capsule, troche, dispersion, suspensions, or solutions.

4. The method according to claim 1 wherein bupropion is administered orally.

5. The method according to claim 1 wherein the amount administered is from about 10 mg to about 750 mg.

6. The method according to claim 5 wherein the amount administered is from about 50 mg to about 600 mg.

7. The method according to claim 6 wherein the amount administered is from about 60 mg to about 450 mg.

8. The method according to claim 1 wherein bupropion is administered as the hydrochloride salt.

9. The method according to claim 1 wherein bupropion is administered in a sustained or controlled release formulation.

10. The method according to claim 1 wherein the pharmaceutically acceptable salt of bupropion is (±)-bupropion hydrochloride.

11. The method according to claim 1 wherein the pharmaceutically acceptable salt of bupropion is (±)-bupropion maleate.

12. The method according to claim 1 wherein the bupropion is (−)-bupropion or pharmacologically acceptable salts thereof.

13. The method according to claim 12 wherein the amount of (−)-bapropion or a pharmaceutically acceptable salt thereof is greater than approximately 90% by weight of the total amount of bupropion.

14. The method according to claim 12 wherein the amount of (−)-bupropion or a pharmaceutically acceptable salt thereof, substantially free of its (+)-stereoisomer, is administered together with a pharmaceutically acceptable carrier.

15. The method according to claim 12 wherein (−)-bupropion is administered as the hydrochloride salt.

16. The method of claim 1 wherein (−)-bupropion is administered in a sustained or controlled release formulation.

17. Use of bupropion or pharmacologically acceptable salts thereof for the preparation of a medicament useful for treating restless legs syndrome in a patient.

18. The use of claim 17, wherein the compound is (±)-bupropion hydrochloride.

19. The use of claim 17, wherein the compound is (±)-bupropion maleate.

20. The use of claim 17, wherein the compound is (−)-bupropion or pharmacologically acceptable salts thereof.
